(19) United States
(12) Patent Application Publication
(10) Pub. No.: US 2025/0025220 A1
(43) Pub. Date: Jan. 23, 2025

(54) CUTTING DEVICE FOR BONE FIXING PLATE

(71) Applicant: Hankil Tech Medical Co., Ltd., Gyeonggi-Do (KR)

(72) Inventors: Jeong-Yup KIM, Gyeonggi-do (KR); Dong-Chan JANG, Gyeonggi-do (KR)

(21) Appl. No.: 18/743,560

(22) Filed: Jun. 14, 2024

(30) Foreign Application Priority Data
Jul. 21, 2023 (KR) 10-2023-0094998

(51) Int. Cl. A61B 17/88; A61B 17/80; B23D 29/02

(52) U.S. Cl. CPC A61B 17/8863; A61B 17/8085; B23D 29/026

(57) ABSTRACT

A cutting device for a bone fixing plate having a multipurpose bite for easily bending bone in the shape of a hook while reducing the size of a fixing plate for fixing fractured bone to each other. Curved surface cutting and V-shape cutting can be performed using one cutting device, thereby reducing the number of equipment, shortening surgery time, and generating a strong holding force at the time of bending to enable precise cutting.

### CROSS-REFERENCE TO RELATED APPLICATIONS

This application claims priority to and the benefit of Korean Patent Application No. 10-2023-0094998 filed in the Korean Intellectual Property Office on Jul. 21, 2023, the entire contents of which are incorporated herein by reference.

### TECHNICAL FIELD

This disclosure relates to a cutting device for a bone fixing plate, and more particularly, to a cutting device for a bone fixing plate having a multipurpose bite for easily bending a bone fixing plate in the shape of a hook while reducing the size of a fixing plate for fixing fractured bone to each other.

### BACKGROUND

When a bone is fractured by an impact, an accident, or the like, the fractured portions are fixed to be in contact with each other through surgery, and a fixing plate is coupled to the bone so as to maintain the fixed state.

For various bone fractures, the use of orthopedic plates is a technique to stabilize bone as needed for proper healing. Generally, a plate, which is a rigid metal, is placed on the outer surface of the bone across the fracture and orthopedic screws extend through the plate and into the bone on both sides of the fracture. The plate supports and provides stability to the bone during the healing period.

However, human bones differ in size, shape, and angle from person to person or from race to race, so it is desirable to use a fixing plate that fits the surgical site during fracture surgery. In actual surgery, the plate is often cut to change the length according to the position and shape of the bone, or the distal end of the plate is bent so that it is well attached to the bone. In this case, a device for cutting the plate is used and a separate device is required for bending the bone fixing plate.

As illustrated in FIGS. 1-4, a fixing plate 10 for fixing bones has a structure in which a plurality of coupling holes 12 are formed in a plate body 11 extending according to the length, position, and angle of bones. A bone screw for coupling 30 is fitted into the coupling hole 12 to fix the plate 10 to the bone.

In the fixing plate 10 as shown in FIG. 1, a protrusion part 13 and a recessed part 14 are alternately formed on the outer surface to form protrusions and recesses. Such protrusions and recesses may allow a cutting device to securely hold the fixing plate 10 when the fixing plate 10 is cut or bent.

In some situations, the end of the fixing plate 10 to be fixed to the bone is cut into a V-shape and used. That is, when it is difficult to attach the fixing plate 10 to a small bone piece, the hook-shaped fixing plate 10 is used. Since it is difficult to provide all the shapes and sizes needed for all bones in the surgical set, during surgery the doctor modifies an existing fixing plate 10 according to the size and shape needed by cutting the plate several times to form it into a hook-shaped plate.

To form the shape illustrated in FIG. 2, the distal end of the plate is cut during surgery. The end cut in the V-shape is referred to as a cut part 15. The cut end is referred to as a segment 20.

In addition, as shown in FIG. 3, the cut part 15 may be bent at a predetermined angle with respect to the plate body 11 to form a bent part 16, and as shown in FIG. 4, the bent part 16 is inserted into a gap between bones or a cutting space of the bones so that the fixing plate 10 can be firmly fixed to the bone.

As described above, during a surgical operation, the fixing plate 10 is often cut to an appropriate length and bent at an appropriate angle, and since separate medical equipment is required for cutting and bending, the number and types of equipment are increased, thereby causing inconvenience.

Furthermore, since the process of modifying the fixing plate requires multiple pieces of equipment, this prolongs the length of the surgery.

### SUMMARY

The disclosed embodiments provide a cutting device for a bone fixing plate that can simultaneously bend an end into a curved surface to change a length of a fixing plate and can also cut the end of the fixing plate into a V shape to form a hook.

In addition, disclosed is a cutting device for a bone fixing plate which has a space having the same shape as the outer shape of the fixing plate and grasps the side of the fixing plate to securely grasp the plate with a small force so as not to be shaken when bending the fixing plate 10.

In an embodiment the cutting device for a bone fixing plate provides a dedicated instrument that can accommodate bone fixing plates of different shapes or sizes so a surgeon can conveniently cut and bend the plate into the proper size and shape in the operating room during surgery.

In an embodiment, the cutting device for a bone fixing plate to be fixed to a bone includes: a handle 102 having two bodies that are rotatably connected to each other by a third hinge coupling part 110; a connecting part 104 having rear ends of the two bodies that are hinge-coupled to one end of the two bodies of the handle 102 by a second hinge coupling part 108, and front ends of the two bodies that are hinge-coupled to each other by a first hinge coupling part 106; first and second cutting parts 114 and 116 that are formed at ends of the connecting part 104 having the two bodies, respectively, and cut the fixing plate 10 into a predetermined shape; and an elastic member 112 that is provided between the two bodies of the handle 102 and generates elastic force that is spaced apart from the handle 102 at a predetermined interval in a state in which no grip force is applied thereto.

In an embodiment, a first bite 118 is detachably installed on the first cutting part 114, and the first bite 118 comprises:
a first plane body 118a in which at least one first bolt coupling hole 118b is formed; a first vertical body 118c extending from one edge of the first plane body 118a; a first curved surface cutting blade 118e formed on one side of the first vertical body 118c in a circular shape; a first curved surface cutting protrusion 118d protruding from a center of the first curved surface cutting blade 118e and inserted into a coupling hole 12 formed on the fixing plate 10 during a cutting operation; a first V-shaped cutting blade 118g formed at the remaining portion of the first vertical body 118c where the first curved cutting blade 118e is not formed; and a first V-shaped cutting protrusion 118f formed outside the first V-shaped cutting blade 118g.

In an embodiment, a second bite 120 is detachably installed on the second cutting part 116, and the second bite 120 comprises:
a second plane body 120a having at least one second bolt coupling hole 120b; a second curved surface cutting blade 120d formed on one side surface of the second plane body 120a in a structure corresponding to the first curved surface cutting blade 118e; a second V-shaped cutting blade 120f formed on the remaining portion of the second plane body 120a where the second curved surface cutting blade 120d is not formed; and a second V-shaped cutting protrusion 120e formed on a side surface of the second V-shaped cutting blade 120f.

The cutting part which cuts the metallic bone fixing plate 10 is formed at an upper edge of the first curved cutting blade 118e and an upper edge of the first V-shaped cutting blade 118g.

In an embodiment, the diameter of the second curved surface cutting blade (120d) is relatively larger than that of the first curved surface cutting blade (118e), and a cutting part processed to cut the metal plate (10) is formed at an edge of the second curved surface cutting blade (120d) and the second V-shaped cutting blade (120f).

The cutting device can perform for both curved cutting and V-shaped cutting with a single cutting device, reducing the number of devices required and shortening the length of the operation. Additionally, the cutting device generates strong gripping force during bending to facilitate precise cutting operations.

The technical solutions obtainable from the present disclosure are not limited to the foregoing solutions, other solutions will be clearly understood by those skilled in the art from the description below.

### BRIEF DESCRIPTION OF THE DRAWINGS

Subject matter of the present disclosure is described in detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a perspective view illustrating a structure of a conventional fixing plate;

FIG. 2 is a perspective view illustrating where an end of a fixing plate is cut;

FIG. 3A is a perspective view illustrating where a cut part of a fixing plate is bent, and FIG. 3B is a side view thereof;

FIG. 4 is a perspective view illustrating fixing plate fixed to a fractured portion of a bone;

FIG. 5 is a perspective view illustrating a structure of a cutting device according to an embodiment;

FIG. 6 is a perspective view illustrating a structure of the cutting device;

FIG. 7 is a perspective view illustrating the entire structure of a first bite;

FIG. 8 is a plan view illustrating a side structure of the first bite;

FIG. 9 is a perspective view illustrating the entire structure of a second bite;

FIG. 10 is a plan view illustrating a side structure of the second bite;

FIG. 11 is a perspective view illustrating a plate is seated at a curved surface cutting position;

FIG. 12 is a perspective view illustrating a plate is seated in a V-shaped cutting position;

FIG. 13 is a plan view simultaneously illustrating two situations where the plate is seated at the curved surface cutting position or the V-shaped cutting position;

FIGS. 14(a) and 14(b) are plan views illustrating a structure of a curved surface cut plate and a V-shaped cut plate, respectively;

FIG. 15 is a perspective view illustrating a structure of a portion gripping the plate vertically; and

FIG. 16 is a perspective view showing where the plate is gripped vertically.

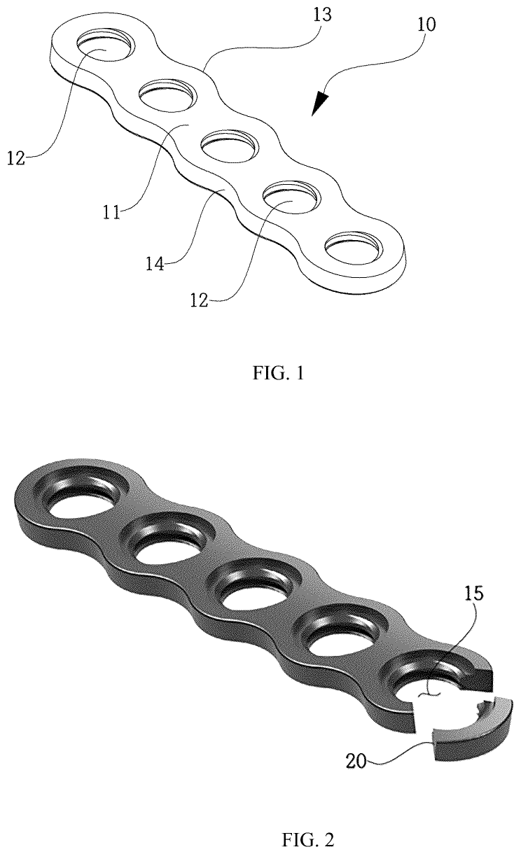

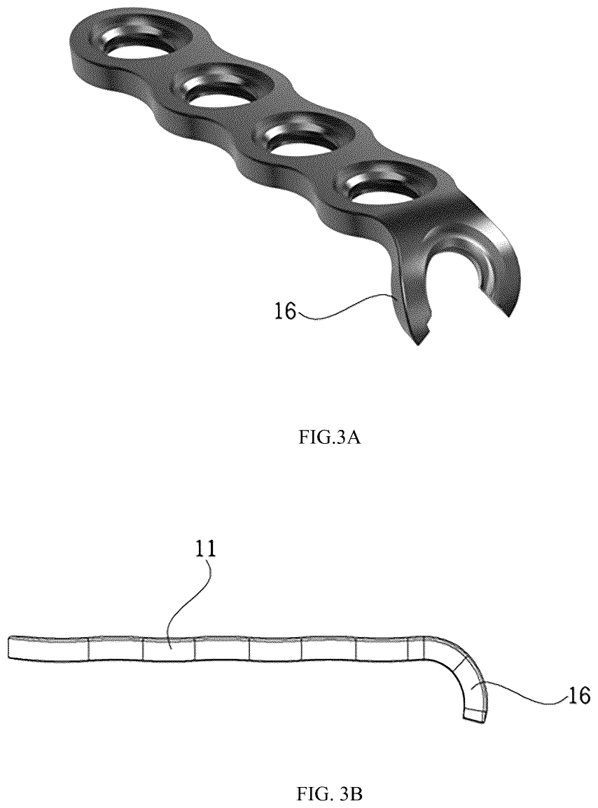

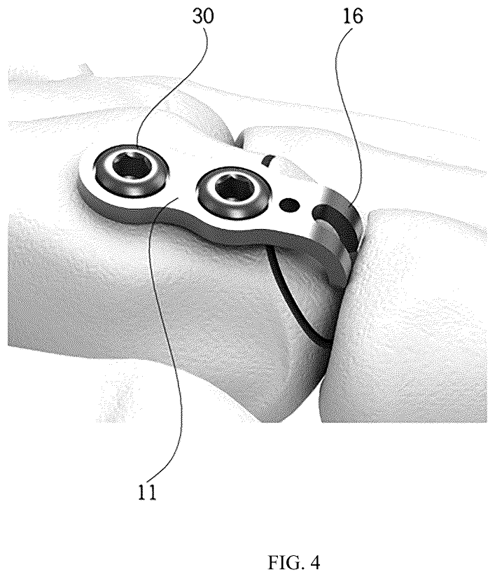

### DETAILED DESCRIPTION

Various embodiments of the disclosure now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

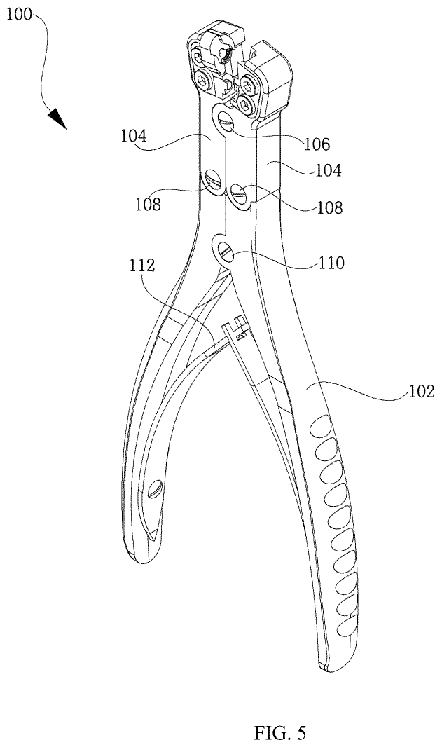

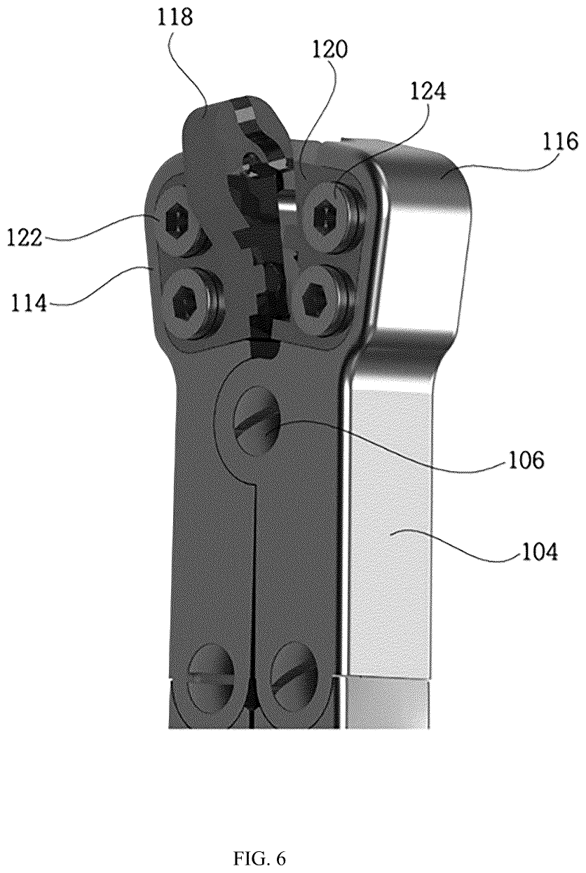

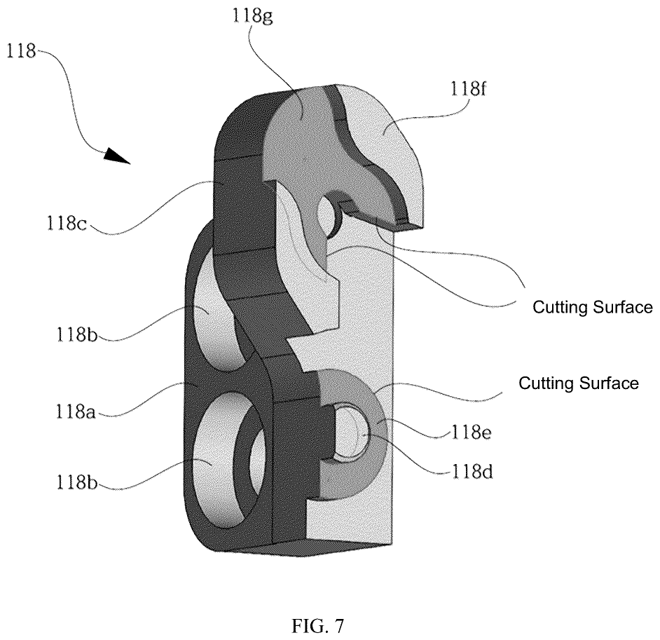

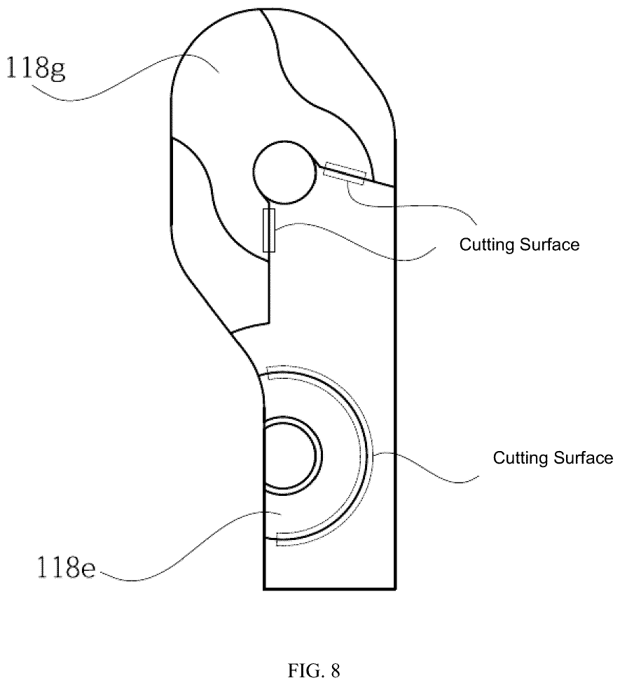

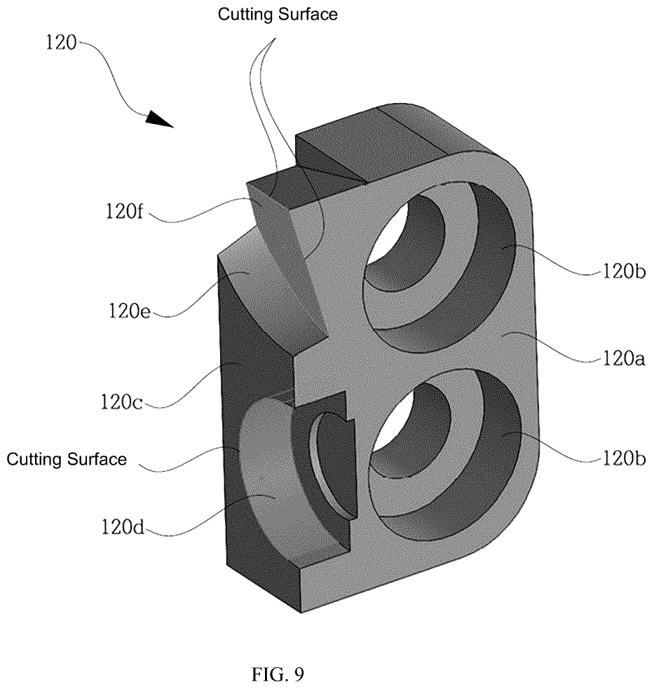

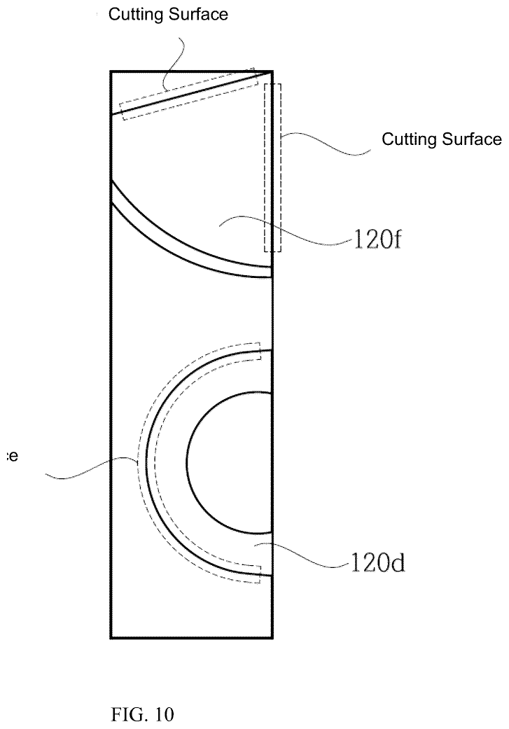

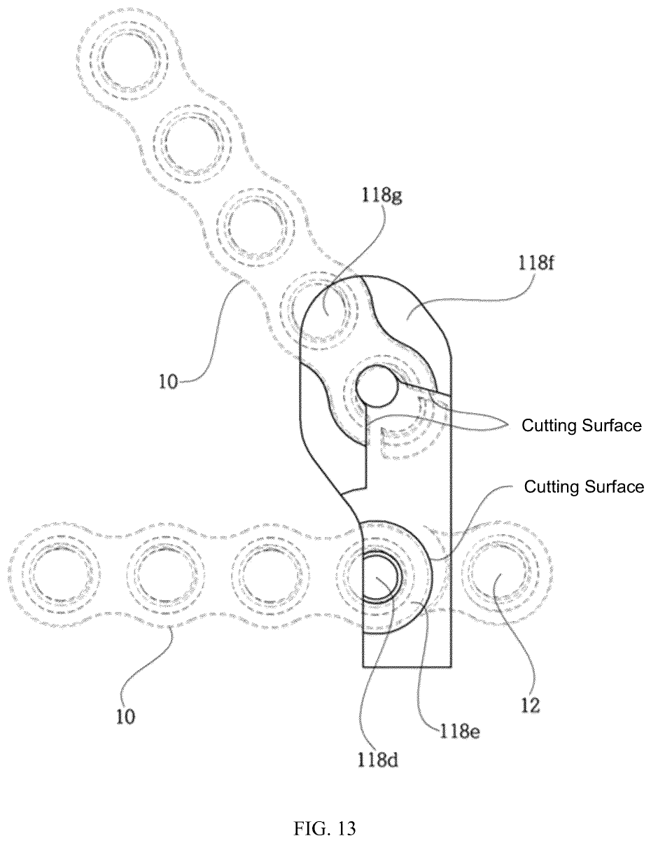

As illustrated in FIGS. 5 through 14, in an embodiment, the cutting device for cutting or bending a fixing plate to be fixed to a bone includes: a pair of handles 102 rotatably coupled to each other by a third hinge 110; a connecting part 104 having rear ends hinge-coupled to the ends of the pair of handles 102 by a second hinge 108 and front ends hinge-coupled to each other by a first hinge 106; a first cutting part 114 and a second cutting part 116 respectively formed at ends of the connecting part 104 to cut the fixing plate 10 into a predetermined shape; and an elastic or spring member 112 disposed between the two bodies of the handle 102 to generate an elastic force to space the handles 102 apart from each other to a predetermined distance when grasping force is not applied to the handles 102.

In an embodiment, curved surface cutting and V-shape cutting can be performed using one cutting device, thereby reducing the number of pieces of equipment, shortening surgery time, and generating a strong holding force at the time of bending or cutting to enable a precise cutting work.

According to an embodiment, the cutting device 100 is made of metal such as stainless steel or titanium, or hard plastic, and has a bite capable of cutting the bone fixing plate 10 made of metal such as titanium. In addition, the double ronguer or double action ronguer method is provided so that a large pressure and cutting force may be generated by applying a small force to the handles 102.

The cutting device 100 includes a handle 102 and a connecting part 104, and a cutting unit is provided at an end of the connecting part 104. The cutting unit is composed of the first cutting part 114 and the second cutting part 116.

The handle 102 is configured such that two bodies facing each other can be rotated by a rotary shaft, and the connecting part 104 rotates the cutting unit as the distance between the bodies becomes larger or smaller according to the rotation of the handle 102. The interval, angle, and distance of the connecting part 104 are changed while the rotation axis at both ends and the rotation axis at the center relatively rotate. This is referred as double ronguer or double action ronguer method.

The connecting part 104 is configured to extend rearward by the same length while allowing the rear ends of the cutting unit to be in close contact with each other between the cross sections facing each other. The connecting part 104 is configured such that the cutting unit is extended rearward, and the cutting unit side end is configured such that a gap is increased toward the front end of the face of the cutting unit and the gap is decreased rearward, and the face is hinged at a portion where the gaps between the face ends meet.

Cross-sections facing each other with respect to the hinged portion are in close contact with each other.

The first hinge coupling part 106 of the connecting part 104 is configured as a rotating shaft that allows a gap between facing cross-sections of the first cutting part 114 and the second cutting part 116 to be narrowed or widened.

The connecting part 104 extends rearward at a predetermined length while being in close contact with each other.

The handle 102 is rotatably shaft-coupled to the rear ends of both sides of the connecting part 104. That is, the handle 102 is configured such that one end thereof is hinge-coupled to the rear end of the connecting part 104, and a second hinge coupling part 108 coupled to the connecting part 104 is formed to be in close contact with the surfaces facing each other for a predetermined length, and the handle 102 is gripped while allowing the gap to be widened outwardly.

The handle 102 is configured such that cross sections of portions of the second hinge coupling part 108 coupled to the connecting part 104, which are in close contact with each other, are rotatably connected to each other, thereby forming the third hinge coupling part 110. The handle 102 has a predetermined length such that the cross sections facing each other rearward from the third hinge coupling part 110 gradually widen outward. However, the gap between two bodies of the handle 102 is formed to a degree that can be gripped with one hand.

In order to easily grip the handle 102, grooves or protrusions for preventing slipping may be formed on the outer surfaces of both bodies of the handle 102.

According to an embodiment, the elastic member 112 is provided between the facing end surfaces of two bodies of the handle 102, and enables two bodies of the handle 102 to be spaced apart from each other at a predetermined interval at all times and the interval between the bodies of the handle 102 to be narrowed to a predetermined interval by a gripping force.

To this end, the elastic member 112 is formed of a pair of leaf springs, such that one end of each leaf spring is fixed to a cross section of both bodies facing each other of the handle 102, and the other end thereof is connected to a third hinge coupling part 110 side of the handle 102 so as to be prevented from being separated from each other, whereby a gap between two bodies of the handle 102 can be increased by a predetermined width. In other words, the elastic member 112 returns to its original state when the grip force between the grips 102 is released.

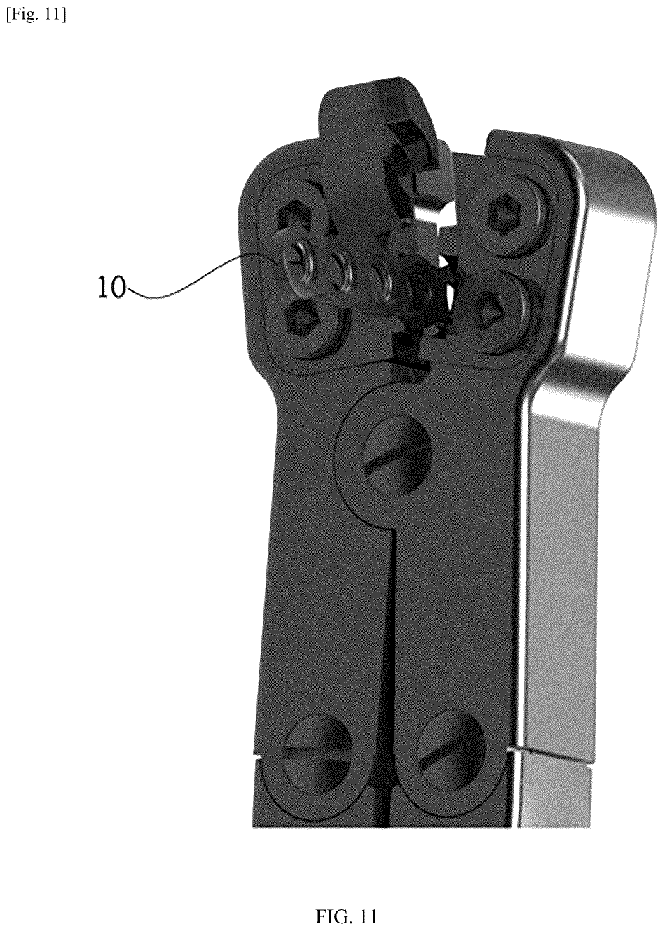

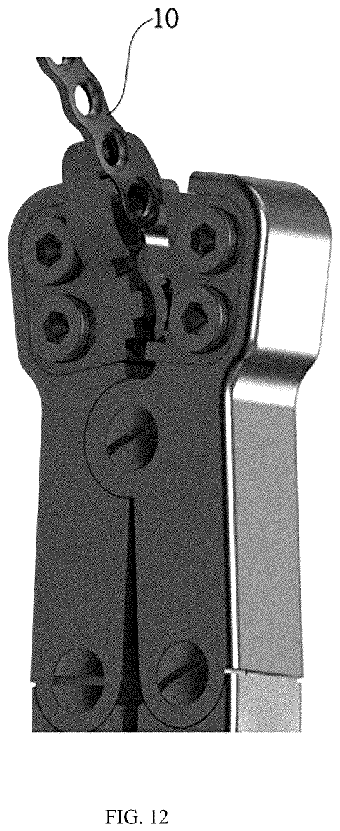

In an embodiment, the interval between the two bites of the cutting unit is maximally widened by the elastic force of the elastic member 112. That is, the two bites included in the cutting unit are maximally spaced apart from each other by the elastic member 112, and in this state, the distal end of the fixing plate 10 to be cut is inserted through the widened portion of the cutting unit. In other words, as shown in FIGS. 11 and 12, the handle 102 is held with one hand, and the fixing plate 10 is inserted between the open bites of the cutting unit.

The cutting unit includes a first cutting part 114 and a second cutting part 116. The first cutting part 114 is provided with a first bite 118, and the second cutting part 116 is provided with a second bite 120. Cutting surfaces of the first bite 118 and the second bite 120 are installed to face each other, and the fixing plate 10 is inserted and seated between the two bites. The first bite 118 and the second bite 120 are detachably coupled to the first cutting part 114 and the second cutting part 116 by a first coupling bolt 122 and a second coupling bolt 124, respectively. Since the type of the fixing plate 10 to be processed may vary depending on the patient's body condition, the cutting device 100 having various sizes of bites is used. A surgeon performing a surgery may be able to replace the bite by unfastening the coupling bolt onsite.

When the operator presses the handle 102 during surgery, the connecting part of the handle 102 rotates about the third hinge coupling part 110, and the distance between the two second hinge coupling parts 108 decreases. As a result, the two arms constituting the connecting part 104 are rotated about the first hinge coupling part 106, and thus the distance between the two bites is decreased. When the operator applies a force while the two bites are in close contact, the fixing plate 10 is cut by pressure.

The first bite 118 and the second bite 120 are configured to be paired with each other, and a portion in which a seating part in a diagonal direction is formed such that the fixing plate 10 is obliquely seated at an end of the cutting unit is referred to as the first bite 118.

The first bolt coupling holes 118b are formed in the first plane body 118a constituting the first bite 118. A first coupling bolt 122 is inserted into the first bolt coupling hole 118b to couple the first bite 118 to the first cutting part 114.

A first vertical body 118c extending at a right angle is formed at one edge of the first flat body 118a. The first flat body 118a and the first vertical body 118c generally extend at a right angle, but the angle therebetween may be greater or less than 90 degrees. In one embodiment, the first V-shaped cutting blade 118g formed on the first vertical body 118c is designed to be bent at an angle of 45 degrees in order to minimize the length of protruding from a side surface.

A first curved cutting blade 118e is formed to protrude in a cylindrical shape on one side of the first vertical body 118c, and the planar shape becomes a part of a circle. In addition, a first curved cutting protrusion 118d protrudes from the center of the first curved cutting blade 118e. The first curved cutting blade 118e is used to cut the fixing plate 10 as much as a circle or a portion of a circle, and is formed to have a diameter as much as a cutting target of a distal end or a middle portion of the fixing plate 10. In addition, the first curved cutting protrusion 118d is inserted into the coupling hole 12 formed in the fixing plate 10 during the cutting process, and fixes the fixing plate 10 so as not to be shaken during the cutting process. In one embodiment, the upper edge of the first curved cutting blade 118e is machined sharp as to cut metal.

A first V-shaped cutting blade 118g is formed at the remaining portion of the first vertical body 118c where the first curved cutting blade 118e is not formed. A first V-shaped cutting protrusion 118f is formed outside the first V-shaped cutting blade 118g. One side of the first V-shaped cutting blade 118g is cut in a V-shape, and the upper edge of the cut portion is sharply processed to cut the metal plate. The angle of the cut portion may be variously changed, and a hook is formed at the distal end of the fixing plate 10 as much as the angle of the cut portion.

It is preferable that the side surface of the first V-shaped cutting protrusion 118f is formed with the protrusion part 13 and the recess part 14 in the same manner as the outer side surface of the fixing plate 10. Accordingly, the fixing plate 10 may be firmly seated on the first V-shaped cutting blade 118g.

Similarly to the above description, second bolt coupling holes 120b are formed in the second flat body 120a of the second bite 120. A second coupling bolt 124 is inserted into the second bolt coupling hole 120b to couple the second bite 120 to the second cutting part 116.

A second side surface 120c is formed at a side of the second flat body 120a, and two cutting blades are formed at the second side surface 120c.

First, a second curved cutting blade 120d is formed on the second side surface 120c. The second curved cutting blade 120d has a structure corresponding to the first curved cutting blade 118e, and is cut in the same circular shape as the shape of the coupling hole in a state in which the expected cutting surface of the plate is seated between the two cutting blades. Since the diameter of the second curved surface cutting blade 120d, which is a partial shape of a circle, is slightly larger than the diameter of the first curved surface cutting blade 118e, when two bites are engaged, the fixing plate 10 may be cut while the first curved surface cutting blade 118e enters the inner space of the second curved surface cutting blade 120d.

The fixing plate 10 seated between the first curved cutting blade 118e and the second curved cutting blade 120d is cut into a curved surface having a larger diameter while having the same shape as the coupling hole 12. As a result, the distal end of the fixing plate 10 has the same circular shape as the shape of the coupling hole 12.

A second V-shaped cutting blade 120f is formed at the remaining portion of the second side surface 120c where the second curved cutting blade 120d is not formed. A second V-shaped cutting protrusion 120e is formed in a vertical direction on a side surface of the second V-shaped cutting blade 120f. The edge of the V-shaped cutting blade 120f also has a cutting portion that is sharply processed to cut the metal plate. The cutting portion is disposed on two straight lines having a predetermined angle, and an angle between the two straight lines is the same as an angle between two cutting portions formed on the first V-shaped cutting blade 118g.

When the second bite 120 is engaged with the fixing plate 10 seated on the first bite 118, the cutting portion of the first V-shaped cutting blade 118g and the cutting portion of the second V-shaped cutting blade 120f overlap each other, and the distal end of the plate 10 is cut in a V-shape. In addition, the fixing plate 10 seated between the first V-shaped cutting blade 118g and the second V-shaped cutting blade 120f is cut in a V-shape in which one side edge of the coupling hole 12 has a predetermined angle to have a hook shape. The operator may bend the hook portion to firmly fix it to the bone.

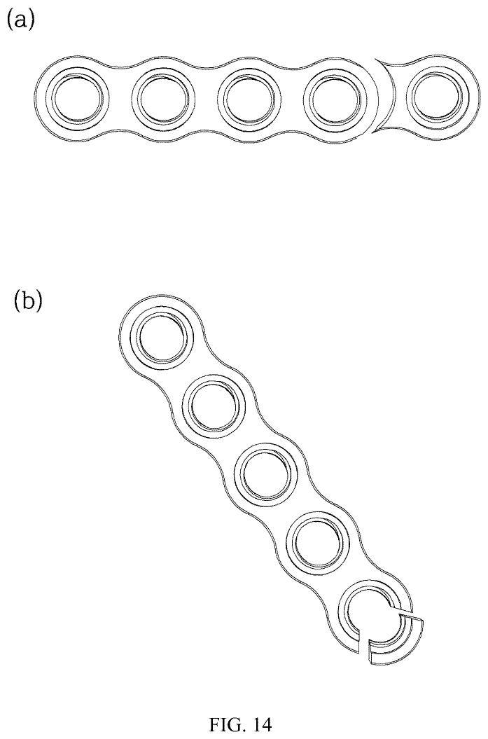

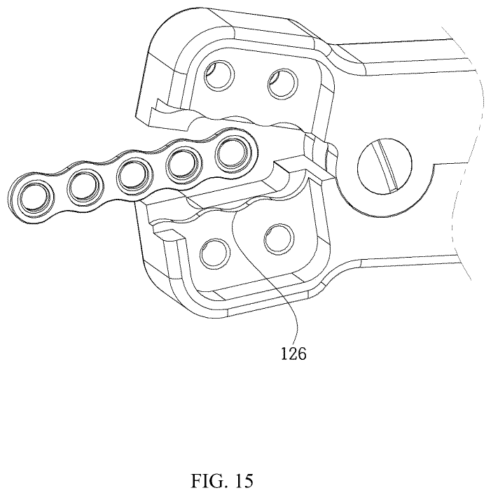

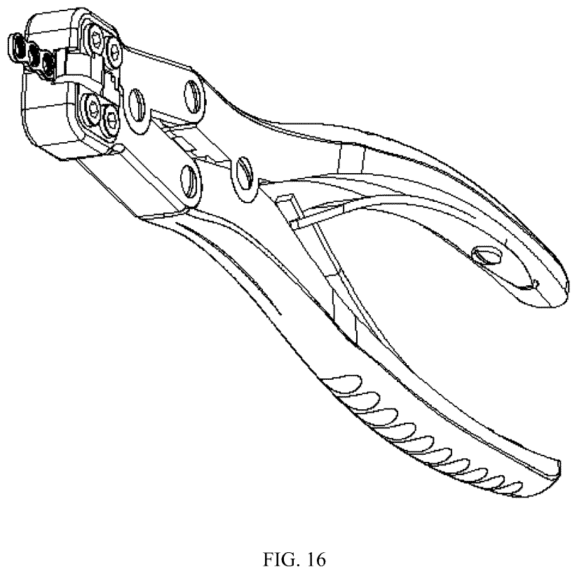

FIG. 14 shows both a curved-surface cut and a V-shaped cut.

Particularly, as shown in FIGS. 11 and 12, the fixing plate 10 is seated in a vertical direction at the side of the cutting unit in the cutting of the curved surface, and is seated in an oblique direction at the side of the cutting unit in the cutting of the V-shape. However, in some cases, the direction in which the fixing plate 10 is seated may be opposite to each other, or all of them may be diagonal directions.

In addition, when cutting a curved surface, it is possible to cut according to the location of the coupling hole such that the length can be freely adjusted according to the intention of an operator. However, in the V-cutting, the end may be cut due to the shape of the bite having an oblique structure.

A fixing groove 126 having the same curve as the outer shape of the fixing plate 10 is formed on the contacting surfaces of the first cutting part 114 and the second cutting part 116 on which the first bite 118 and the second bite 120 are mounted, respectively. The fixing groove 126 have the same shape as the left and right side surfaces of the fixing plate 10, and come into close contact with the side surface of the fixing plate 10 in a state in which the first bite 118 and the second bite 120 are in contact with each other. When the first bite 118 and the second bite 120 are engaged with each other in a state in which the plane of the plate 10 is erected to face the left and right directions, the fixing plate 10 is seated at the fixing groove 126 on the side surfaces thereof, and thus, even when the fixing plate 10 is pulled in the outer longitudinal direction of the bite, the fixing plate 10 is not separated due to the fixing groove 126 in close contact with the curved surfaces of the left and right sides of the fixing plate 10. As a result, even if the operator's grip on the handle 102 is weakened, the fixing plate 10 does not fall out during cutting or bending.

In one embodiment, a general grip method may be applied in which the plane of the fixing plate 10 faces up and down to contact the planes of the first bite 118 and the second bite 120.

Although preferred embodiments of the disclosure have been described with reference to the accompanying drawings, it will be understood by those skilled in the art that the technical configuration described above may be implemented in other specific forms without changing the technical spirit or essential features of this disclosure. Therefore, it should be understood that the embodiments described above are exemplary in all aspects and are not limited thereto, and the scope of the disclosure is indicated by claims described below rather than the detailed description, and it should be interpreted that the meaning and scope of claims and all changes or modified forms derived from the equivalent concept thereof are included in the scope of the disclosure.

### DESCRIPTION OF REFERENCE NUMERALS

10: Fixing plate
11: Plate body
12: Coupling hole
13: Protrusion part
14: Recess part
15 Cut part
16: Bent part
20: Segment
30: Bone screw for coupling
100: Cutting device
102: Handle
104: Connecting part
106: First hinge coupling part
108: Second hinge coupling part
110: Third hinge coupling part
112 Elastic member
114: First cutting part
116: Second cutting part
118: First bite
120: Second bite
122: First coupling bolt
124: Second coupling bolt
126: Fixing groove

What is claimed is:

1. A device for cutting and/or bending a bone fixing plate to be fixed to a bone, the device comprising:
a pair of handles rotatably connected to each other by a third hinge;
a connecting part having a rear end that is hinge-coupled to an upper end of the pair of handles by a second hinge, and having front ends that are hinge-coupled to each other by a first hinge;
first and second cutting units disposed on the front ends of the connection parts for cutting the fixing plate into a predetermined shape; and
an elastic member extending the pair of handles that urges that pair of handles apart from each other.

2. The device of claim 1, further comprising:
a first bite detachably installed on the first cutting part, the first bite comprising:
a first plane body having at least one first bolt coupling hole;
a first vertical body extending from one edge of the first plane body;
a first curved surface cutting blade formed in a circular shape on one side of the first vertical body;
a first curved surface cutting protrusion protruding from a center of the first curved surface cutting blade which is adapted to be inserted into a coupling hole of the fixing plate during a cutting operation;
a first V-shaped cutting blade formed at a portion of the first vertical body where the first curved cutting blade is not formed; and
a first V-shaped cutting protrusion formed outside the first V-shaped cutting blade.

3. The device of claim 2, further comprising:
a second bite detachably installed on the second cutting part, the second bite comprising:
a second plane body having at least one second bolt coupling hole;
a second curved surface cutting blade formed on one side surface of the second plane body in a structure corresponding to the first curved surface cutting blade;
a second V-shaped cutting blade formed at a portion of the second plane body where the second curved surface cutting blade is not formed; and
a second V-shaped cutting protrusion formed on a side surface of the second V-shaped cutting blade.

4. The device of claim 3, further comprising:
a cutting surface formed at an upper edge of the first curved cutting blade and an upper edge of the first V-shaped cutting blade.

5. The device of claim 4, wherein a diameter of the second curved surface cutting blade is larger than a diameter of the first curved surface cutting blade, and the cutting surface is formed at an edge of the second curved surface cutting blade and the second V-shaped cutting blade.
